(19) United States
(12) Patent Application Publication
(10) Pub. No.: US 2011/0258713 A1
(43) Pub. Date: Oct. 20, 2011

(54) COMPOSITIONS AND METHODS FOR RE-PROGRAMMING CELLS WITHOUT GENETIC MODIFICATION

(75) Inventors: Yong Zhu, Anaheim, CA (US); Shili Wu, Edmond, OK (US); Jun Bao, Rowland Heights, CA (US)

(73) Assignee: VIVOSCRIPT, INC., Costa Mesa, CA (US)

(21) Appl. No.: 13/141,326

(22) PCT Filed: Dec. 23, 2009
(86) PCT No.: PCT/US09/69518

§ 371 Date: Jun. 22, 2011

Related U.S. Application Data
(60) Provisional application No. 61/203,438, filed on Dec. 23, 2008.
(60) Provisional application No. 61/210,586, filed on Mar. 19, 2009.
(60) Provisional application No. 61/216,511, filed on May 18, 2009.
(60) Provisional application No. 61/226,659, filed on Jul. 17, 2009.

(51) Int. Cl. A01K 67/033; C12N 5/04; C12N 5/07; C12N 5/071; C12N 5/0783; C12Q 1/02; C07K 14/435; A61K 38/17; A61P 35/00; A61P 3/00; A61P 29/00; A61P 9/00; A61P 25/00; A61P 37/00; A61P 3/10; A61P 25/28; A61P 25/16; A61P 7/06; A61P 19/02; A61P 17/02; A61P 17/00; A61P 1/16; A61P 27/16; A61P 1/04; A61P 21/04; A61P 25/18; A61K 49/00; A61M 37/00; C12N 1/00; B82Y 5/00

(52) U.S. Cl. 800/9; 435/243; 435/410; 435/375; 435/377; 435/29; 530/409; 530/402; 514/21.2; 514/19.2; 514/19.3; 514/16.4; 514/17.7; 514/6.9; 514/17.8; 514/13.5; 514/16.8; 514/18.7; 514/16.6; 514/7.3; 514/18.6; 514/13.2; 424/9.2; 604/500; 977/902

(57) ABSTRACT

The present inventions are directed to compositions and methods regarding the reprogramming of biological samples (such as cells) without introducing exogenous genes to the samples. In particular, the present inventions are directed to transducible materials that are capable of transducing into the biological samples but are not genes or causing genetic modifications. The present inventions also are directed to methods of reprogramming the path of biological samples or treating diseases using the tranducible compositions thereof.

### CROSS-REFERENCE TO RELATED APPLICATIONS

The present application claims priority to U.S. Provisional Application 61/203,438, filed Dec. 23, 2008; U.S. Provisional Application 61/210,586, filed Mar. 19, 2009; U.S. Provisional Application 61/216,511, filed May 18, 2009; and U.S. Provisional Application 61/226,659, filed Jul. 17, 2009, all of which are incorporated herein by reference in their entirety.

### BACKGROUND OF THE INVENTION

Embryonic stem cells are capable of differentiating into many types of cells of human body. The majority of somatic cells are terminally differentiated and were believed to lack the capability of changing to other types of somatic cells. Recent advances in induced pluripotent stem cell (iPSC) and transdifferentiation fields have changed this paradigm. Somatic cells can be reprogrammed to induced pluripotent stem cell (iPSC), i.e. via ectopic expression of four transcription factors, i.e. Oct4 (e.g. SEQ ID NO: 1), Sox2 (e.g. SEQ ID NO: 2), K1f4 (SEQ ID NO: 3), and cMyc (e.g. SEQ ID NO: 4) via viral transduction (Okita et al., 2007; Takahashi and Yamanaka, 2006). A number of modified genetic approaches were further developed to produce iPSCs with potentially reduced risks, including using non-integrating adenoviruses to deliver reprogramming genes (Stadtfeld et al., 2008), transient transfection of reprogramming plasmids (Okita et al., 2008), a piggyBac transposition system and Cre-excisable viruses (Soldner et al., 2009; Woltjen et al., 2009). Furthermore, strategies of exploiting endogenous gene expression in certain cell types also allowed easier reprogramming and/or with less required exogenous genes (Aasen et al., 2008; Kim et al., 2008; Shi et al., 2008b). Moreover, small molecules have been identified that enhance reprogramming efficiency and replace certain reprogramming factors (Huangfu et al., 2008a; Huangfu et al., 2008b; Li et al., 2009; Shi et al., 2008a; Shi et al., 2008b). However, all of those methods to date still involve the use of genetic materials with drawbacks of introducing unknown, unwanted, or even harmful genome modifications by exogenous sequences in target cells and having inadequate control over expression levels of transgenes. To address these drawbacks, there are needs in the field to reprogram cells without relying upon or introducing exogenous genetic materials such as exogenous genes or DNA fragments or vector containing exogenous DNA or genes.

### BRIEF SUMMARY OF THE INVENTION

One aspect of the present disclosure relates to a transducible material comprising an effector domain. The effector domain is capable of exerting reprogramming changes of a biological sample once transduced into a biological sample. In certain embodiments, the effector domain is inherently capable of transducing into the biological sample.

In certain embodiments, the transducible material further comprises a transduction domain which is covalently or non-covalently associated with or linked to the effector domain. In certain embodiments, the transduction domain is covalently linked to the effector domain through a linker.

In certain embodiments, the transducible material is capable of selectively transducing into one or more specific biological samples or becoming transducible in a specific environment surrounding the biological sample.

Another aspect of the present disclosure relates to a composition comprising a biological sample and a transducible material, wherein the transducible material has transduced into the biological material.

Another aspect of the present disclosure relates to a method of reprogramming a biological sample by exposing the biological sample to a composition comprising a transducible material.

Another aspect of the present disclosure relates to a method of treating a disease or condition in a biological organism comprising administering a pharmaceutical composition comprising a transducible material into the biological organism.

Another aspect of the present disclosure relates to a method of developing cell-based therapies for various diseases or conditions comprising the step of reprogramming an iPSC, an embryonic stem cell, or a progenitor cell to a transplantable somatic cell or a transplantable progenitor cell using a transducible material.

Another aspect of the present disclosure relates to a method of developing disease models comprising the step of reprogramming an iPSC, an embryonic stem cell, or a progenitor cell to a transplantable somatic cell or a transplantable progenitor cell using a transducible material.

Another aspect of the present disclosure relates to a method of identifying an effector domain comprising covalently or non-covalently associating a test effector domain to a transduction domain to form a test transducible molecule, exposing the test molecule to a biological sample, and measuring a reprogramming level of the biological sample.

### BRIEF DESCRIPTION OF THE DRAWINGS

FIG. 1: Characterization of transducible materials (I). (A) Schematic of protein expression vector for transducible materials Oct4-11R (SEQ ID NO: 12), Sox2-11R (SEQ ID NO: 13), K1f4-11R (SEQ ID NO: 14), and cMyc-11R (SEQ ID NO: 15), Linker: SEQ ID NO: 55; Effector Domain: Oct4 (SEQ ID NO: 1), Sox2 (SEQ ID NO: 2), K1f4 (SEQ ID NO: 3), or cMyc (SEQ ID NO: 4). (B) Stability of the four transducible materials (Oct4-11R , Sox2-11R, K1f4-11R, and cMyc-11R) under the cell culture condition examined by Western blot analysis.

FIG. 2: Characterization of transducible materials (II). Protein transduction of 11R-tagged transducible materials into OG2-MEF cells examined by immunocytochemistry. Oct4: MEF cells transuded with Oct4-11R (green), Sox2: MEF cells transuded with Sox2-11R (red), K1f4: MEF cells transuded with K1f4-11R (red) and cMyc: MEF cells transuded with cMyc-11R (green). DAPI: Cells stained with DAPI to visualize the nuclei (blue) and the images were merged.

FIG. 3: Characterization of transducible materials (III). Protein induced pluripotent stem (piPS) cells clonally expanded and self-renewed in chemical defined media and feeder free condition.

FIG. 4: Generation of piPS cells by transducible materials Oct4-11R, Sox2-11R, K1f4-11R, and cMyc-11R. (A) Timeline of piPS cell generation. (B) Oct4-GFP+ piPS cell colonies initially observed around day 30-35. Phase: representative phase contrast image; and GFP: fluorescence image. (C) Oct4-GFP+ piPS cells sustained long term self-renewal under conventional mESC growth condition. (D) The long-term expanded piPS cells grew as compact and domed colonies that expressed strong ALP, a typical pluripotency marker. (E) piPS cells expressed other typical pluripotency markers, examined by immunofluorescence: SEA-1 (red), Sox2 (red), Oct4 (ed) and Nanog (red). DAPI: DAPI staining for visualization of the nuclei (blue), and the images were merged. (F) RT-PCR analysis of endogenous pluripotency gene expression in piPS cells. (G) Oct4 promoter methylation analysis by bisulfite genomic sequencing. Open and closed circles indicate unmethylated and methylated CpGs, respectively.

FIG. 5: In vitro and in vivo pluripotency of piPS cells (I). piPS cells effectively differentiated in vitro into cells in the three germ layers: Tuj1: characteristic TUJ¹+ neuronal cells-ectoderm (red): Bryt: Brachyury+ mesoderm cells (red); and Sox17: Sox17⁺ definitive endoderm cells. Images were merged with DAPI (blue) staining

FIG. 6: In vitro and in vivo pluripotency of piPS cells (II) (A) RT-PCR analysis of in vitro differentiation of piPS cells. (B) Chimeric embryos (13.5 dpc, 2 out of 7, left) obtained after transfer of the piPS cell aggregated embryos into a pseudo-pregnant mouse (top). Such piPS cells contributed to the germline cells (Oct4-GFP positive) in isolated genital ridge tissue from chimeric embryos (bottom).

FIG. 7: Schematic of protein expression vectors for transducible materials. His6: SEQ ID NO: 59; Effector Domain: Ngn3 (SEQ ID NO: 8), PDX1 (SEQ ID NO: 9); MafA (SEQ ID NO: 10), or Foxp3 (SEQ ID NO: 11); Linker: SEQ ID NO: 55.

FIG. 8: Reprogramming of liver and pancreatic exocrine cells to insulin-producing beta cells by transducible materials His6-Ngn3-11R (SEQ ID NO: 30), His6-PDX1-11R (SEQ ID NO: 31) and His6-MafA-11R (SEQ ID NO: 32) in mouse (I). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) Immunofluorescent analysis (IFA) of Mouse-1 liver; B) IFA of Mouse-2 liver; and C) IFA of Mouse-3 liver.

FIG. 9: Reprogramming of liver and pancreatic exocrine cells to insulin-producing beta cells by transducible materials His6-Ngn3-11R, His6-PDX1-11R and His6-MafA-11R in mouse (II). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) IFA of Mouse-4 liver (1); B) IFA of Mouse-4 liver (2); C) IFA of Mouse-5 liver (1); D) IFA of Mouse-5 liver (2); E) IFA of Mouse-6 liver (1); and F) IFA of Mouse-6 liver (2).

FIG. 10: Reprogramming of liver and pancreatic exocrine cells to insulin-producing beta cells by transducible materials His6-Ngn3-11R, His6-PDX1-11R and His6-MafA-11R in mouse (III). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) IFA of Mouse-1 pancrease; B) IFA of Mouse-2 pancrease (1); C) IFA of Mouse-2 pancrease (2); and D) IFA of Mouse-3 pancrease.

FIG. 11: Reprogramming of liver and pancreatic exocrine cells to insulin-producing beta cells by transducible materials His6-Ngn3-11R, His6-PDX1-11R and His6-MafA-11R in mouse (IV). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) IFA of Mouse-4 pancrease (1); B) IFA of Mouse-4 pancrease (2); C) IFA of Mouse-5 pancrease (1); D) IFA of Mouse-5 pancrease (2); and E) IFA of Mouse-6 pancrease.

FIG. 12: Reprogramming of T cells to Treg cells by transducible material His6-Foxp3-11R (SEQ ID NO: 33) (IA). Flow cytometric analysis of CD4 and CD25 protein expression in PBMC with lacking of CD14 monocytes: isotope control, PBS control, sample buffer control and protein (BSA 100 μg/ml) control.

FIG. 13: Reprogramming of T cells to Treg cells by transducible material His6-Foxp3-11R (IB). Flow cytometric analysis of CD4 and CD25 protein expression in PBMC with lacking of CD14 monocytes treated with His16-Foxp3-11R of 10 μg/ml, 20 μg/ml, or 50 μg/ml.

FIG. 14: Reprogramming of T cells to Treg cells by transducible material His6-Foxp3-11R (IIA). Flow cytometric analysis of CD4 and CD25 protein expression in PBMC: isotope control, and PBS control.

FIG. 15: Reprogramming of T cells to Treg cells by transducible material His6-Foxp3-11R (IIB). Flow cytometric analysis of CD4 and CD25 protein expression in PBMC: sample buffer control and protein (BSA 100 μg/ml) control.

FIG. 16: Reprogramming of T cells to Treg cells by transducible material His6-Foxp3-11R (IIC). Flow cytometric analysis of CD4 and CD25 protein expression in PBMC treated with His16-Foxp3-11R of 10 μg/ml, or 50 μg/ml.

FIG. 17: Reprogramming of T cells to Treg cells by transducible material His6-Foxp3-11R (IID). Flow cytometric analysis of CD4 and CD25 protein expression in PBMC treated with His16-Foxp3-11R of 100 μg/ml.

### DETAILED DESCRIPTION OF THE INVENTION

One aspect of the present disclosure relates to a transducible material comprising an effector domain.

In certain embodiments, a transducible material used herein refers to a material or a molecule which is not DNA or derived from DNA but is capable of crossing or transducing or being crossed through a membrane of a biological sample (e.g., a cell membrane) so that the transducible material can enter or be brought into the inside of the biological sample from the outside of the biological sample and exerts reprogramming efforts. For example, the transducible material may interact with cell-surface receptors which facilitate the entry of the material into cells through receptor mediated endocytosis.

In certain embodiments, a transducible material is a selective transducible material which is more likely to transduce into a specific type of biological samples (e.g. cancer or tumor cells) or becomes transducible in a specific microenvironment in or around a biological sample (e.g. microenvironment around cancer or tumor) than other biological samples. For example, the selective transducible material comprises a transduction domain (e.g. a cell-targeting peptide or an activatable cell penetrating peptide) that preferably delivers the selective transducible material into a specific type of biological sample or become transducible in a microenvironment around a biological sample.

Without being bounded to any theories, it is contemplated that the transducible materials may cross a cell membrane and enter into cytoplasm to reprogram cytoplasm activities such as translation, post-translation modification, signaling pathway, apoptosis pathway. It is further contemplated that the transducible material may cross the nucleus membrane and reprogram or modulate DNA or chromosomal replication, gene transcription, and RNA splicing.

An effector domain is a motif or a molecule which, once inside a biological sample, is capable of exerting reprogramming changes of the biological sample. The effector domain may interact with molecules (e.g., proteins, DNA, RNA, sugars, and lipids) in the biological sample (e.g., in cytoplasm or nuclei) and lead to changes such as proliferation, differentiation, dedifferentiation, transdifferentiation, retrodifferentiation, transdertermination, apoptosis, and morphogenesis. The effector domain can be 1) a polypeptide, or a fragment or a mimic thereof; 2) a polynucleotide which cannot be gene expressed once transduced or incorporated into the genome of the biological sample or cause genetic modification but nevertheless interacts with molecules in the biological sample (e.g., a ribozyme, an antisense molecule, a siRNA or miRNA, an oligonucleotide, and the like); and 3) a small molecule or other chemical compound (e.g. chemotherapy drugs).

In certain embodiments, an effector domain is inherently transducible, e.g. PDX1 (e.g. SEQ ID NO: 9) and NeuroD (e.g. SEQ ID NO: 7).

One example of the effector domain is a polypeptide such as a transcription factor, a chromosome remodeling protein, an antibody, or a fragment or mimic thereof. Another example of the effector domain is a small molecule which is not a polymer and binds with a biolpolymer such as protein, nucleic acid, or polysaccharide and alters the activity or function of the biopolymer. Examples of small molecules include, without limitation, acetylation inhibitors, transcription activators, signal pathway activators, signal pathway inhibitors, and methylation inhibitors.

In another embodiment, an effector domain can be at least one polypeptide that reprograms a somatic cell into a stem cell or change a cell state from one to another. For example, the effector domain can be 1) a polypeptide selected from the group consisting of K1f4 (e.g. SEQ ID NO: 3), Sox2 (e.g. SEQ ID NO: 2), Lin28 (e.g. SEQ ID NO: 5), Oct4 (e.g. SEQ ID NO: 1), cMyc (e.g. SEQ ID NO: 4), Nanog (e.g. SEQ ID NO: 6), and any combination thereof; 2) a polypeptide selected from the group consisting of K1f4, Sox2, Oct4, cMyc, and any combination thereof; 3) a polypeptide selected from the group consisting of Sox2, Oct4, Lin28, Nanog, and any combination thereof; 4) a polypeptide selected from the group consisting of Ngn3 (e.g. SEQ ID NO: 8), PDX1 (e.g. SEQ ID NO: 9), MafA (e.g. SEQ ID NO: 10), NeuroD (e.g. SEQ ID NO: 7), and any combination thereof; 5) a polypeptide comprising Foxp3 (e.g. SEQ ID NO: 11); 6) a polypeptide selected from the group consisting of Oct4, Sox2, K1f4, Lin28, Nanog, cMyc, Ngn3, PDX1, MafA, NeuroD, Foxp3, and any combination thereof; 7) a combination of polypeptides Oct4, Sox2, K1f4 and cMyc; and 8) a combination of polypeptides Ngn3, PDX1 and MafA.

In certain embodiments, a transducible material further comprises a transduction domain. A transduction domain is a motif that is capable of facilitating the entry of the transducible material into a biological sample (e.g., a cell). The transducible domain is associated with the effector domain covalently, noncovalently or via a linker. In certain embodiments, the transduction domain is covalently linked to the effector domain through a linker. In certain embodiments, the linker is a glycine-rich linker that comprises one or more glycine residues (e.g. esggggspg (SEQ ID NO: 55)).

Examples of a transduction domain include, without limitation, polymers such as cationic lipid polymers and nanoparticles, protein transduction domains (PTD), cell penetrating peptides (CPP1), cell permeating peptides (CPP2), activatable cell penetrating peptides or conjugates (ACPP), and cell-targeting peptides (CTP).

CPP1, CPP2, and PTD are peptides known to facilitate delivery of a molecular cargo associated thereof into cells. The association between a CPP1, CPP2 or PTD and the molecular cargo can be through covalent bond or non-covalent interactions. The molecular cargo can be small chemical molecules, peptides, protein, fragment of DNA, RNA such as siRNA and miRNA, or nanosize particles. For example, CPP1 and PTD include 5 to 20 amino acid peptide motifs that are capable of penetrating cells independent of surface transporters and of cell cycle phase. CPP1 and PTD can also be capable of penetrating through blood—brain barriers. CPP1 and PTD can deliver proteins and peptides in vitro and in vivo with uniform distribution throughout the organism after parenteral administration. Cationic PTDs can act as nuclear localization signals and carry an associated molecular cargo to cell nuclei. Examples of protein transduction domains include, without limitation, TAT (e.g. YGRKKRRQRRR, SEQ ID NO: 34), poly-arginine (e.g. poly-arginine having 7-11 arginine residues such as RRRRRRR, RRRRRRRR, RRRRRRRRR, RRRRRRRRRR (SEQ ID NO: 35)and RRRRRRRRRRR (SEQ ID NO: 36)), Penetratin (Antennapedia, e.g. RQIKIWFQNRRMKWKK (SEQ ID NO: 38)), VP22 (e.g. DAATATRGRSAASRPTQRPRAPARSASRPRRPVQ (SEQ ID NO: 39)), Transportan (e.g. GWTLNSAGYLLGKINLKALAALAKKIL (SEQ ID NO: 40)), MAP (e.g. KLALKLALKALKAALKLA (SEQ ID NO: 41)), MTS (e.g. AAVALLPAVLLALLP (SEQ ID NO: 42)), PEP-1 (e.g. KETWWETWWTEWSQPKKKRKV (SEQ ID NO: 43)), Arg/Trp analogue (e.g. RRWRRWWRRWWRRW (SEQ ID NO: 44)), polyguanidine peptoids (e.g. polyguanidine peptoids with a 6-methylene spacer between backbone and guanidine group such as N-arg 5, 7 or 9 peptoids), HIV-1 Rev (e.g. SEQ ID NO: 60), Flock house virus coat peptide (e.g. SEQ ID NO: 61), and DNA-binding peptides such as c-Fos (e.g. SEQ ID NO: 62), c-Jun (e.g. SEQ ID NO: 63)and yeast GCN4 (e.g. SEQ ID NO: 64).

Cell-targeting peptides are proteins or peptides that bind to cell-surface receptors and enter cells through endocytosis. In certain embodiments, a cell-target peptide targets specific tissues or cell types, for example, GnRH peptides (e.g. SEQ ID NO: 58) target biological samples that express GnRH receptors (e.g. solid tumors and hormone-responsive cancer cell lines). More examples of cell-targeting peptides and the specific biological samples targeted are listed in Table 1.

TABLE 1
Examples of cell-targeting peptides and
the specific biological samples targeted.

| Sequence | | | Targeted tissue or cells |
|---|---|---|---|
| ID No. | Peptide sequence | Length | and the cellular targets |
| SEQ ID NO: 45 | TSPLNIHNGQKL | 12 | Human head and |
| | | | neck solid tumors |
| SEQ ID NO: 46 | CGKRK | 5 | Tumor neovasculature, |
| | | | Heparan sulfate |
| SEQ ID NO: 47 | CGNKRTRGC | 7 | breast carcinoma |
| SEQ ID NO: 48 | SMSIARL | 7 | Prostate vasculature |
| SEQ ID NO: 49 | FQHPSFI | 7 | hepatocellular carcinoma |
| | | | cell line |
| | RGD | 3 | Integrin receptor′, αVβ3 |
| | NGR | 3 | Tumor neovasculature, |
| | | | Amino-peptidase N |
| SEQ ID NO: 50 | VHSPNKK | 7 | Endothelial VCAM-1 |
| | | | expressing cells; |
| | | | VCAM-1 |
| SEQ ID NO: 51 | RRPYIL | 6 | Adenocarcinoma cells; |
| | | | Neurotensin receptor |
| SEQ ID NO: 52 | EDYELMDLLAYL | 12 | Various carcinoma |
| SEQ ID NO: 53 | LTVSPWY | 7 | breast carcinoma; erbB2 |
| SEQ ID NO: 54 | ATWLPPR | 7 | Tumor neovasculature; |
| | | | VEGF receptor |

An activatable cell penetrating peptide or conjugate (ACPP) comprises a cationic CPP1, CPP2 or PTD and a neutralizing anionic counterpart. In certain embodiments, the cationic CPP1, CPP2 or PTD and the anionic counterpart are associated via noncovalent interactions (e.g. charge-charge interaction) and/or covalent cleavable linker (e.g. matrix metaloprotease (MMP) cleavable sequence). Transduction of an ACPP into cells are inhibited until the noncovalent interactions are disrupted and/or the cleavable linker is cleaved. For example, without being bound to any theory, the anionic counterparts comprise one or more pH sensitive groups such as sulfonamide groups, which are protonated at pH 7.4 (the pH in the blood stream) and become neutral at slightly acidic pH (e.g. pH 6.8). Therefore, charge-charge interactions between cationic CPP1, CPP2 or PTD and the anionic counterpart can be interrupted in slightly acidic microenvironment (e.g. in or around tumor or cancer). MMP concentration in blood stream is lower than that in a microenvironment around tumor or cancer. Therefore, MMP cleavable sequence, which is not cleaved in the bloodstream, is cleaved in environment around tumor or cancer. The cationic CPP1, CPP2 or PTD is no longer neutralized by the anionic counterpart, and therefore is exposed to facilitate the translocation into cells (e.g. tumor or cancer cells). In certain embodiments, the CPP1, CPP2 or PTD is TAT. In certain embodiments, the anionic counterparts comprise pH-sensitive polymer (e.g. di-block copolymer) comprising pH-sensitive groups (e.g. sulfonamide groups).

For another example, an activatable cell penetrating conjugate comprises a conventional hydrophobic core made of a polymer into which an effective domain is incorporated, a peripheral hydrophilic layer composed of poly-ethylene glycol and one or more cationic CPP1s, CPP2s or PTDs, and one or more anionic counterpart that neutralize the cationic CPP12, CPP2s or PTDs through charge-charge interactions. Such charge-charge interactions are expected to shield the cationic charges during delivery until the transduction material reaches a slightly acidic microenvironment (e.g. tumor or cancer), which triggers protonation of the anionic counterparts and disrupts the charge-charge association. Subsequently the cationic CPP1s, CPP2s, or PTDs, previously quenched by the anionic counterpart, are now capable of facilitating delivery of the effector domain into the surrounding cells (e.g. tumor or cancer cells).

In certain embodiments, a selective transducible material comprises a transduction domain selected from the group consisting of cell-targeting peptides and activatable cell penetrating peptides and activatable cell penetrating conjugates.

A transduction domain is associated to an effector domain via covalent bond, non-covalent interactions or through a linker. Thus a transducible material can be made by obtaining the transduction domain and the effector domain separately and associate together through a covalent bond or non-covalent interactions (e.g., repulsive interactions, dipole interactions, hydrogen bonding interactions, dispersive interactions, charge-charge interactions, solvent, counter ion and entropic effects, and water and hydrophobic effects). In certain embodiments, the transduction material is prepared by mixing the effector domain and transducible domain. Alternatively, a transducible material can be produced by isolating the material from natural resources or recombinantly. In the case when both domains are peptides or polypeptides, the effector domain can be linked to the N-terminus or C-terminus of the transduction domain and the transducible polypeptide can be made synthetically through chemical synthesis or recombinantly through recombinant technology.

In certain embodiments, a transducible material comprises an effector domain that is inherently transducible, and a transduction domain associated with the effector domain via covalent or non-covalent interactions.

In certain embodiments, a transducible material further comprises one or more motifs that do not interrupt the function of the effector domain or the transduction domain. In certain embodiments, these motifs are linked covalently, non-covalently or through a linker to the effector domain and/or the transduction domain. In certain embodiments, these motifs facilitate the preparation and/or purification of the transducible material. One example of such motif is a polyhistidine-tag to facilitate protein purification in preparation of the transducible material. In certain embodiments, the polyhistidine-tag comprises at least six histidine residues (e.g. MGSSHHHHHHSSGLVPRGSH (“His6,” SEQ ID NO: 59)).

In certain embodiments, a transducible material includes, for example, Oct4-11R (SEQ ID NO: 12), Sox2-11R (SEQ ID NO: 13), K1f4-11R (SEQ ID NO: 14), Lin28-11R (SEQ ID NO: 16), Nanog-11R (SEQ ID NO: 17), cMyc-11R (SEQ ID NO: 15), Ngn3-11R (SEQ ID NO: 19), PDX1-11R (SEQ ID NO: 20), MafA-11R (SEQ ID NO: 21), NeuroD-11R (SEQ ID NO: 18), and Foxp3-11R (SEQ ID NO: 22), wherein 11R (SEQ ID NO: 37) stands for a polyarginine sequence of 11 arginine residues linking to a linker through which the polyarginie sequence is covalently linked to the effector domain. In certain embodiments, a transducible material includes, for example, His6-Oct4-11R (SEQ ID NO: 23), His6-Sox2-11R (SEQ ID NO: 24), His6-K1f4-11R (SEQ ID NO: 25), His6-Lin28-11R (SEQ ID NO: 27), His6-Nanog-11R (SEQ ID NO: 28), His6-cMyc-11R (SEQ ID NO: 26), His6-Ngn3-11R (SEQ ID NO: 30), His6-PDX1-11R (SEQ ID NO: 31), His6-MafA-11R (SEQ ID NO: 32), His6-NeuroD-11R (SEQ ID NO: 29), and His6-Foxp3-11R (SEQ ID NO: 33).

In certain embodiments, a transducible material can be combined with one or more adjuvants such as growth factors to stimulate cellular growth. For example, islet growth factor (e.g. betacellulin) is used as an adjuvant in reprogramming liver and/or pancreatic exocrine cells to insulin producing cells (e.g. (3 cells).

Another aspect of the present disclosure relates to a composition comprising a biological sample and at least one transducible material, wherein the transducible material has transduced into the biological sample. For example, the composition includes a transducible material comprising Foxp3 (e.g. Foxp3-11R and His6-Foxp3-11R) and a T cell wherein the transducible material has transduced into the T cell; a composition includes a piPS cell and one or more transducible materials comprising a polypeptide selected from the group consisting of Oct4, K1f4, Sox2 and cMyc, and any combination thereof (e.g. Oct4-11R, K1f4-11R, Sox2-11R, cMyc-11R, His6-Oct4-11R, His6-K1f4-11R, His6-Sox2-11R and His6-cMyc-11R); and a composition including a liver or pancreatic exocrine cell and one or more transducible materials comprising a polypeptide selected from the group consisting of Ngn3, PDX1, MafA, NeuroD, and any combination thereof (e.g. Ngn3-11R, PDX1-11R, MafA-11R, His6-Ngn3-11R, His6-PDX1-11R and His6-MafA-11R) wherein the transducible materials have transduced into the liver or pancreatic exocrine cell.

Another aspect of the present disclosure relates to a method of reprogramming a biological sample by exposing the biological sample to a composition comprising a transducible material. In certain embodiments, the method preferably reprograms a specific type of biological sample (e.g. cancer or tumor cells) or biological samples in or around a specific microenvironment within a biological organism (e.g. microenvironment around cancer or tumor) than other biological samples by exposing biological samples to a composition comprising a selective transducible material.

In one embodiment, a biological sample includes a cell, a cluster of cells, a tissue, an organ, a biological body from a biological organism. The biological sample can be normal, healthy sample or abnormal, diseased sample (e.g., cancer or tumor).

A biological organism includes, for example, a microorganism (e.g., bacteria), a fungus, a plant and an animal (e.g., a human).

An organ from an animal biological organism (e.g., human) includes, for example, a circulatory organ (e.g., heart, blood and blood vessels), a digestive organ (e.g., salivary glands, esophagus, stomach, liver, gallbladder, pancreas, intestines, rectum and anus), an endocrine organ (e.g., endocrine glands such as the hypothalamus, pituitary or pituitary gland, pineal body or pineal gland, thyroid, parathyroids and adrenals, i.e., adrenal glands), an integumentary organ (e.g., skin, hair and nails), a lymphatic organ (e.g., lymph nodes and vessels, tonsils, adenoids, thymus and spleen), a muscular organ (e.g., muscles), a nervous organ (e.g., brain, spinal cord, peripheral nerves and nerves), a reproductive organ (e.g., ovaries, fallopian tubes, uterus, vagina, mammary glands, testes, vas deferens, seminal vesicles, prostate and penis), a respiratory organ (e.g., the pharynx, larynx, trachea, bronchi, lungs and diaphragm), a skeletal organ (e.g., bones, cartilage, ligaments and tendons), a urinary system (e.g., kidneys, ureters, bladder and urethra). An organ can be normal or healthy, and alternatively, abnormal or unhealthy (e.g., cancerous).

An organ from a plant biological organism includes, for example, root, stem, leaf, flower, seed and fruit.

A tissue from a biological sample (e.g. an animal) includes a connective tissue, a muscle tissue, a nervous tissue, and an epithelial tissue. A tissue can be normal or healthy, and alternatively, abnormal or unhealthy (e.g., cancerous). A tissue from a biological sample (e.g. a plant) includes an epidermis, a vascular tissue and a ground tissue.

A cell can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a sertoli cell, a leydig cell, an ovum, an oocyte). A cell can be normal, healthy cell; or a diseased or unhealthy cell (e.g., a cancer cell).

A cell further includes a mammalian stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and an unipotent stem cell (See, Hans R. Schöler (2007). “The Potential of Stem Cells: An Inventory” in Nikolaus Knoepffler, Dagmar Schipanski, and Stefan Lorenz Sorgner. Humanbiotechnology as Social Challenge. Ashgate Publishing, Ltd. pp. 28), any of which may be induced from a somatic cell. A stem cell may also include a cancer stem cell.

In another embodiment, “reprogramming a biological sample” used herein is exchangeable with or refers to modulating, altering, or changing the biological activities of the biological sample (e.g., cell) or modulating, altering, or changing the state or status of the biological sample from one to another. For example, by exposing a biological sample (e.g., a cell) to a transducible material, the biological activities of the cell (e.g., cell growth, cell division, cell metabolism, cell cycle, cell signaling, DNA replication, transcription, RNA splicing, protein synthesis, post-translation modification) are modulated or altered so as to lead to cell proliferation, differentiation (e.g., from progenitor cells to terminally differentiated cells), dedifferentiation (e.g., from terminally differentiated cells to pluripotent stem cells), transdifferentiation (e.g., from one type of terminally differentiated cells to another type of terminally differentiated cells), retrodifferentiation (e.g., from terminally differentiated cells to progenitor cells), transdertermination (e.g., from one type of progenitor cells to a type of terminally differentiated cells that are usually derived from another type of progenitor cells under natural conditions), apoptosis (e.g., cell death of cells or cancer cells), morphogenesis, and changes in the cell fate. For another example, the state of a biological sample can be altered or changed from abnormal or diseased state to normal or healthy state (e.g., from cancer cells to noncancer cells); from one cell type to another cell type (e.g., from undifferentiated stem cells to differentiated stem cells or specialized cells), from differentiated or specialized cells to undifferentiated cells or stem cells (e.g., an omnipotent stem cell, a pluripotent stem cell, a multipotent stem cell, an oligopotent stem cell and an unipotent stem cell)(e.g., from fibroblast cells to induced pluripotent stem cells (iPSCs)), from somatic cells to stem cells or induced stem cells, from one state of stem cells to another state of stem cells (e.g., from ominipotent stem cells to pluripotent stem cells), from one type of differentiated cells to another type of differentiated cells (e.g., T-cells to regulatory T cells, pancreatic exocrine cells to insulin-producing beta cells).

In another embodiment, a biological sample is exposed to a transducible material and reprogrammed. The biological sample can be exposed in vitro, in vivo or ex vivo. For example, the biological sample is exposed in vitro through contacting the sample with the transducible material in an environment outside of a living biological organism (e.g., in a cell culture system or a test tube). The biological sample is exposed in vivo through contacting the material with a biological organism containing the sample or introducing (e.g., through administration) the material into the organism. The transducible materials can be administered via any known administration route such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) route. The biological sample is exposed ex vivo when the biological sample (e.g., a cell, a tissue or an organ) is taken outside the biological organism, contacted with the transducible material, and placed back to the same or different biological organisms. Examples of ex vivo exposures comprise removing a biological sample from the biological organism, exposing the biological sample to a transducible material, and transplanting the biological sample transduced with the transducible material back to the biological organism.

In certain embodiments, OG2-MEF cells are exposed to a composition comprising protein Oct4-11R, Sox2-11R, K1f4-11R and cMyc-11R and reprogrammed to induced pluripotent stem cells (iPSCs).

In certain embodiments, T cells are exposed to a composition comprising protein Foxp3-11R or His6-Foxp3-11R and programmed to regulatory T cells (Treg cells).

In certain embodiments liver and/or pancreatic exocrine cells are exposed to a composition comprising one or more proteins selected from the group consisting of Ngn3-11R, PDX1-11R, MafA-11R, NeuroD-11R, His6-Ngn3-11R, His6-PDX1-11R, His6-MafA-11R, and His6-NeuroD-11R and reprogrammed into insulin producing cells (e.g. β cells). In certain embodiments, the composition further comprises one or more adjuvant such as Islet growth factor (e.g. betacellulin). In certain embodiments, the composition comprises His6-Ngn3-11R, His6-PDX1-11R, and His6-MafA-11R. In certain embodiments, the composition comprises His6-Ngn3-11R, His6-PDX1-11R, His6-MafA-11R and betacellulin. Without bond to the mechanism, it is further contemplated that such reprogramming is through transdetermination and/or transdifferentiation.

Another aspect of the present disclosure relates to a method of treating, preventing or reducing a disease or condition in a biological organism by administering a composition comprising a transducible material into the organism. In certain embodiments, the composition is a pharmaceutical composition comprising a transducible material. In certain embodiments, the composition comprises a selective transducible material. The treatment, prevention or reduction of a disease or condition is associated with the change or reprogramming of a biological sample (e.g., a cell, a tissue or an organ) in the organism.

In certain embodiments, the disease or condition treatable by the method include, without limitations, tumor, cancer, metabolic diseases or conditions (e.g. type I and type II diabetes and obesity), inflammatory conditions, cardiac diseases, neurogenerative diseases (e.g. anemia, amyotrophic lateral sclerosis, spinal cord injury, burns, or arthritis), autoimmune diseases or conditions (e.g. acute disseminated encephalomyelitis (ADEM), Addison's disease, alopecia areata, ankylosing spondylitis, antiphospholipid antibody syndrome (APS), anemia (e.g. autoimmune hemolytic anemia and pernicious anaemia), arthritis, psoriatic arthritis, rheumatoid arthritis, diabetes mellitus type 1, autoimmune hepatitis, autoimmune inner ear disease, bullous pemphigoid, coeliac disease, Chagas disease, chronic obstructive pulmonary disease, Crohns disease, dermatomyositis, endometriosis, Goodpasture's syndrome, Graves' disease, Guillain-Barre syndrome (GBS), Hashimoto's disease, hidradenitis suppurativa, Kawasaki disease, IgA nephropathy, idiopathic thrombocytopenic purpura, interstitial cyctitis, lupus erythematosus, mixed connective tissue disease, morphea, multiple sclerosis (MS), myasthenia gravis, narcolepsy, neuromyotonia, pemphigus vulgaris, psoriasis, polymyositis, primary billiary cirrhosis, schizophrenia, scleroderma, Sjogren's syndrome, stiff person syndrome, temporal arteritis (“Giant cell arteritis”), ulcerative colitis, vasculitis, vitiligo, and Wegener's granulomatosis).

For example, it is contemplated that a transducible material can be administered to a biological organism having a tumor to activate the apoptosis of the tumor cells or make tumor cells more sensitive to chemotherapy, radiotherapy, or cancer drugs.

In certain embodiments, a transducible material can be administered to a biological organism to enhance or attenuate immune system and thus treat or prevent immune-related diseases or inflammatory diseases. For example, protein Foxp3-11R or His6-Foxp3-11R is transduced to T cells and programs them to Treg cells, which suppress the overactive immune system and thus is a treatment for auto-immune diseases

In certain embodiments, a transducible material can be administered to a biological organism to treat metabolic diseases or conditions such as type I diabetes, type II diabetes, or obesity. For example, to treat diabetes, a composition comprising a protein selected from the group consisting of Ngn3-11R, PDX1-11R, MafA-11R, NeuroD-11R, His6-Ngn3-11R, His6-PDX1-11R, His6-MafA-11R, His6-NeuroD-11R and any combination thereof can be transduced into liver and/or pancreatic exocrine cells and programs them to insulin producing cells (e.g. β cells). In certain embodiments, one or more adjuvant such as Islet growth factor (e.g. betacellulin) is/are also administered to the biological organism. In certain embodiments, the composition comprises His6-Ngn3-11R, His6-PDX1-11R, and His6-MafA-11R. In certain embodiments, the composition comprises His6-Ngn3-11R, His6-PDX1-11R, His6-MafA-11R and betacellulin. Without bond to the mechanism, it is further contemplated that such reprogramming is through transdetermination and/or transdifferentiation.

It is further contemplated that a transducible material can be administered to a biological organism to treat cardiac diseases such as myocardial infarction or ischemia.

Another aspect of the present disclosure relates to a method of reprogramming iPSCs, embryonic stem cells, or other types of stem or progenitor cells to certain types of somatic cells or progenitor cells, which can be developed as cell-based therapies for various diseases or conditions, including anemia, neurodegenerative diseases, cancer, amyotrophic lateral sclerosis, spinal cord injury, burns, heart diseases, diabetes, and arthritis. The stem cells or progenitor cells may be patient-specific or non-patient-specific, repaired to rid of molecular defects or not, before they are exposed to transducible materials for controlled differentiation or reprogramming. The reprogrammed cells may be enriched, purified, or manipulated before transplanted back to patients.

Another aspect of the present disclosure relates to a method of reprogramming iPSCs, embryonic stem cells, or other types of stem or progenitor cells to certain types of somatic cells or progenitor cells, which can be used as disease models for drug screening, mechanism study, toxicity assay, or other research and drug discovery and development tools. For example, the method comprises exposing an iPSC, an embryonic stem cell, or a progenitor cell to a composition comprising a transducible material to reprogram the iPSC, embryonic stem cell, or progenitor cell to a transplantable somatic cell or a transplantable progenitor cell; transplanting the transplantable somatic cell or transplantable progenitor cell into a biological sample or a biological organism; developing the biological sample or biological organism to become a disease model. For another example, the method comprises reprogramming patient-specific cells to iPSCs using a transducible materials; further generating different type of cells from patient specific iPSCs with or without tranducible materials; and developing a disease model using patient-specific iPSCs or iPSC-derived cells. For another example, the method of developing drug screening or toxicity models comprises reprogramming somatic cells, progenitor cells, or multipotent cells to iPSCs using a transducible material; further generating different type of cells from iPSCs with or without exposing to transducible materials; and using iPSCs and/or iPSC-derived cells to screen the effects and/or toxicities of different compounds.

Another aspect of the present disclosure relates to a method of developing cell-based therapies for various diseases or conditions comprising the step of reprogramming an iPSC, an embryonic stem cell, or a progenitor cell to a transplantable somatic or progenitor cell using a transducible material; transplanting the transplantable somatic or progenitor cell into a biological sample or biological organism; assessing the therapeutic effect of the transplantable somatic or progenitor cell.

Another aspect of the present disclosure relates to a method of identifying a effector domain, wherein the method comprises the steps of covalently linking a test effector domain to a know transduction domain to form a test transducible molecule; exposing the test molecule to a biological sample, and measuring the reprogramming of the biological sample to indicate whether the test effector domain can exerts a change in the biological sample. It is also contemplated that another aspect of the present disclosure relates to a method of identifying a transducible domain, wherein the method comprises the steps of covalently linking a known effector domain to a test transduction domain to form a test transducible molecule; exposing the test molecule to a biological sample, and measuring the location of the test molecule in or the reprogramming effect of the biological sample to indicate whether the test transduction domain can transduce the effector domain into the biological sample.

### EXAMPLES

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### Example 1

Reprogramming somatic cells to induced pluripotent stem cells (iPSCs)

1.a. Preparation of transducible material Oct4-11R, Sox2-11R, K1f4-11R, and cMyc-11R.

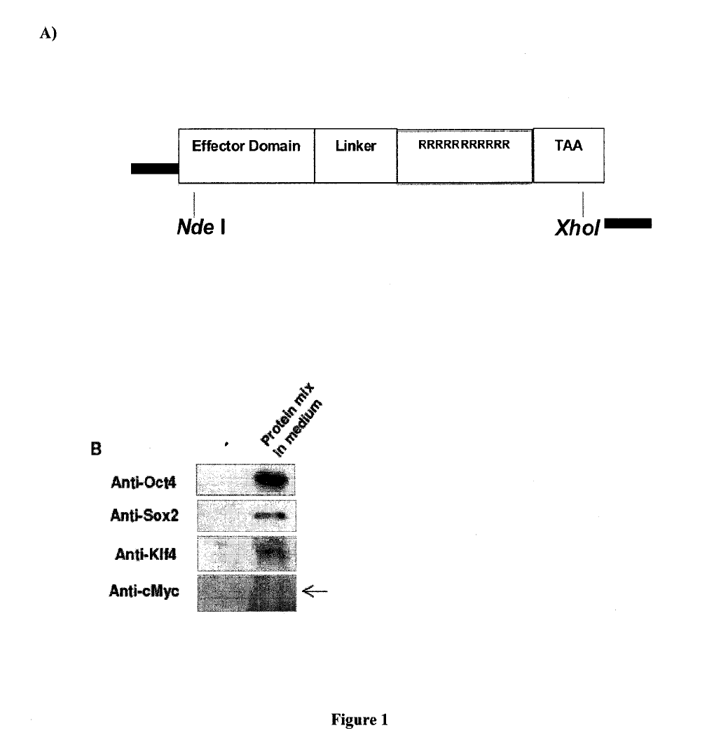

A poly-arginine protein transduction domain was fused to the C-terminal of each reprogramming proteins Oct4, Sox2, K1f4 and cMyc through a linker SEQ ID NO. 55 to form a fused protein Oct4-11R, Sox2-11R, K1f4-11R and cMyc-11R respectively (FIG. 1A). These poly-arginine fused proteins were expressed in E. Coli in inclusion body form, which were then solubilized, refolded, and further purified to render transducible materials Oct4-11R, Sox2-11R, K1f4-11R and cMyc-11R. The protein identities were confirmed by mass spectrometry and Western blot analysis (FIG. 1 B).

1.b. Cell permeability and stability of transducible material Oct4-11R, Sox2-11R, K1f4-11R, and cMyc-11R

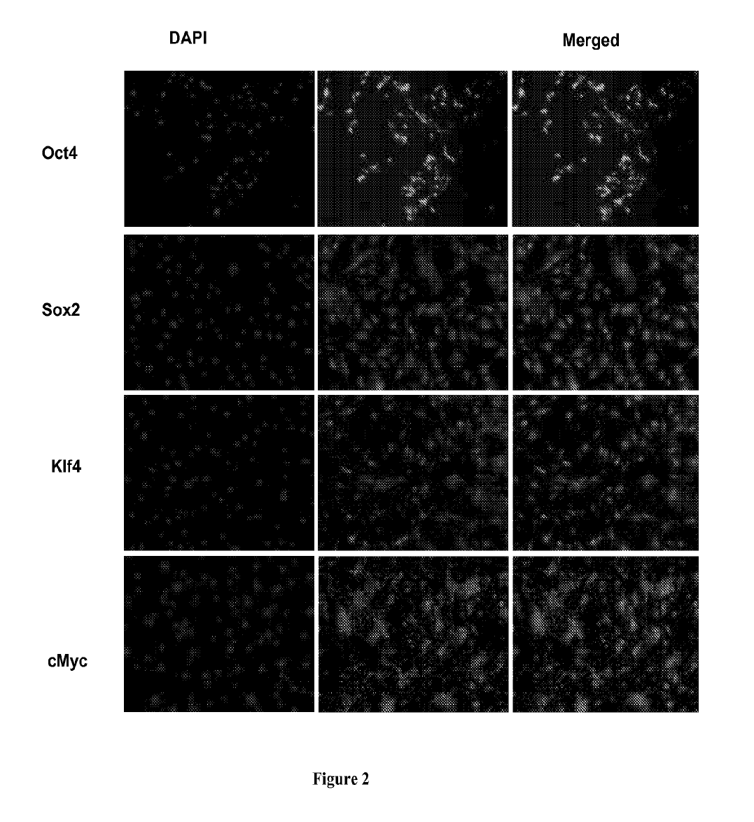

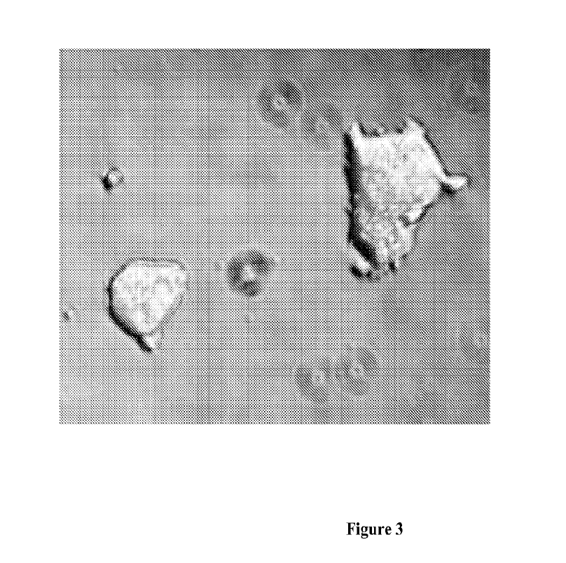

A transducible material (Oct4-11R, Sox2-11R, K1f4-11R, or cMyc-11R) was added to mouse embryonic fibroblast (MEF) cells at various concentrations for 6-72 hours. Cell morphology and protein presence were examined by immunocytochemistry. The transducible materials were found to enter cells at concentrations of 0.5-8 μg/ml within 6 hours, and translocated into nucleus (FIG. 2). In addition, the transduced proteins were fairly stable inside of cells for up to 48 hours (FIG. 3).

1.c. Reprogramming OG2/Oct4-GFP reporter MEF cells.

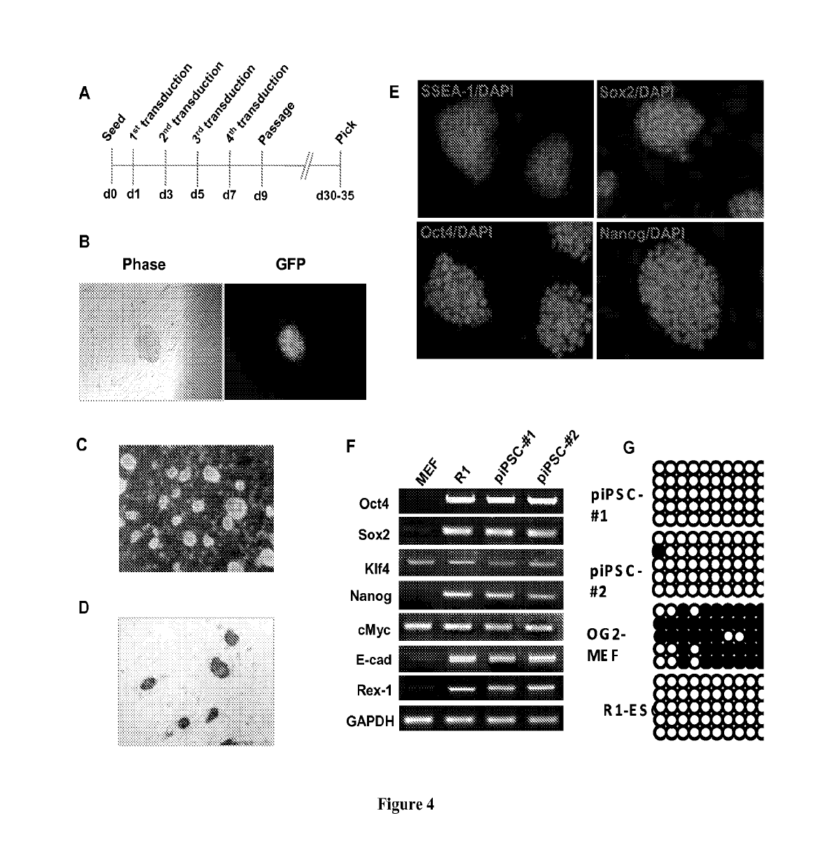

The protein transduction condition described in paragraph 0047 was used to reprogram OG2/Oct4-GFP reporter MEF cells. Cells were treated in 4 cycles. In each cycle the fibroblasts (initially seeded at the density of 5×10⁴ cells/well in a six-well plate) were first treated with transducible materials Oct4-11R, Sox2-11R, K1f4-11R and cMyc-11R at 8 μg/ml in the mESC growth media supplemented with or without 1 mM valproic acid (VPA, a inhibitor of the enzyme histone deacetylase 1 (HDAC1)) for overnight, followed by changing to the same media without the transducible material and VPA, and culturing for additional 36 hours before the next cycle of the treatment. After completing repeated protein transduction of a transducible material, the treated cells were transferred onto irradiated MEF feeder cells and kept in mESC growth media until colonies emerged around day 30-35 (FIG. 4A). 3 GFP+colonies per 5×10⁴ cells were obtained when the cells were transduced with Oct4-11R, Sox2-11R, K1f4-11R, and cMyc-11R and treated with VPA, and 1 GFP+ colony per 5×10⁴ cells were obtained when the cells were transduced with Oct4-11R, Sox2-11R, or K1f4-11R respectively and treated with VPA. Those initial GFP+ colonies were subsequently passaged under conventional mESC growth conditions to yield piPS cells, and were further characterized.

The generated murine piPS cells have been stably expanded for over twenty passages, and were morphologically indistinguishable to classic mES cells, forming compact domed small colonies (FIGS. 4B and 4C). They expressed typical pluripotency markers examined by immunocytochemistry and staining, including ALP (FIG. 4D), Oct4, Nanog, Sox2, and SSEA1 (FIG. 4E). RT-PCR analysis confirmed endogenous gene expression of these pluripotency markers and additional pluripotency genes (FIG. 4F). A single cell survival assay also demonstrated that piPS cells clonally expanded efficiently as Oct4-positive colonies in feeder-free and N2/B27-chemically defined conditions. Furthermore, bisulphite genomic sequencing analyses of the Oct4 promoter revealed that it was demethylated in piPS cells similarly to the mES cells, while the MEFs' Oct4 promoter was hypermethylated (FIG. 4G). This result further suggests a reactivation of the pluripotency transcription program in these piPS cells.

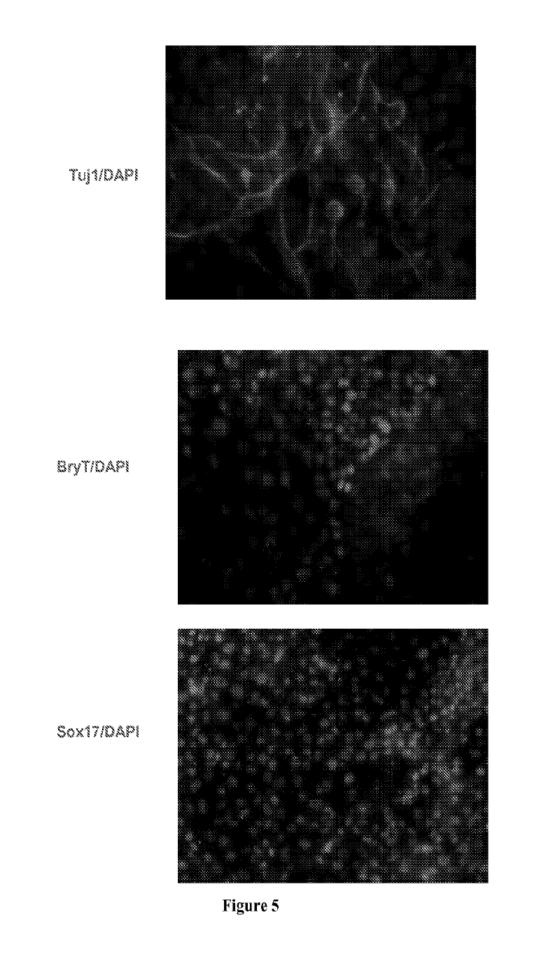

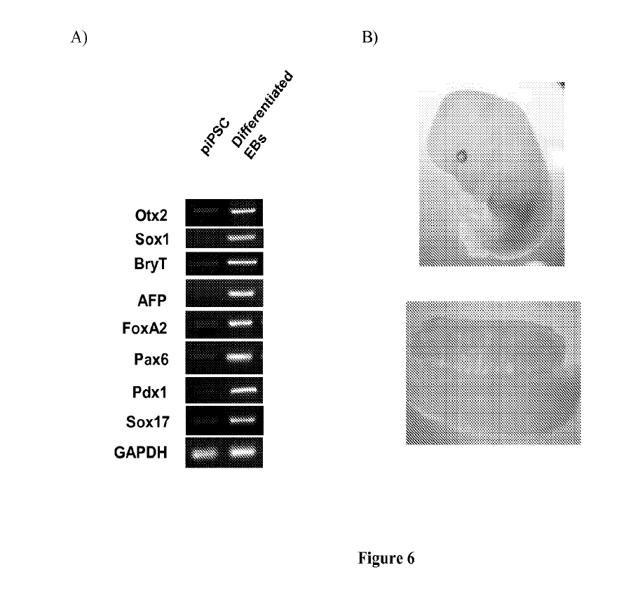

To examine the developmental potential of piPS cells, standard in vitro differentiation using embryoid bodies (EB) or monolayer chemically defined step-wise differentiation, as well as in vivo chimerism assays were performed. piPS cells efficiently formed EB in suspension, and differentiated into cells in the three primary germ layers, including primitive endoderm (AFP, Sox17), foregut endoderm (FoxA2), pancreatic cells endoderm (PDX1, Pax6), mesoderm (Brachyury), and neural (Sox1) and neuronal cells (βIII-tubulin)-ectoderm (FIGS. 5 and 6A). These piPS cells efficiently incorporated into the inner cell mass of a blastocyst following aggregation with an 8-cell embryo, and led to chimerism with germline contribution (FIG. 6B) in vivo after the aggregated embryos were transplanted into mice, as suggested by observation of Oct4-GFP+ cells in the gonad tissue in 2 out of 7 embryos (FIG. 6B bottom). These in vitro and in vivo characterizations collectively confirm that the purified transduction material Oct4-11R, Sox2-11R, K1f4-11R, and cMyc-11R are able to reprogram MEFs to piPS cells, which are morphologically and functionally similar to conventional mES cells.

### Example 2

Reprogramming of liver and pancreatic exocrine cells to insulin-producing beta cells by transducible materials His6-Ngn3-11R, His6-PDX1-11R and His6-MafA-11R in mouse.

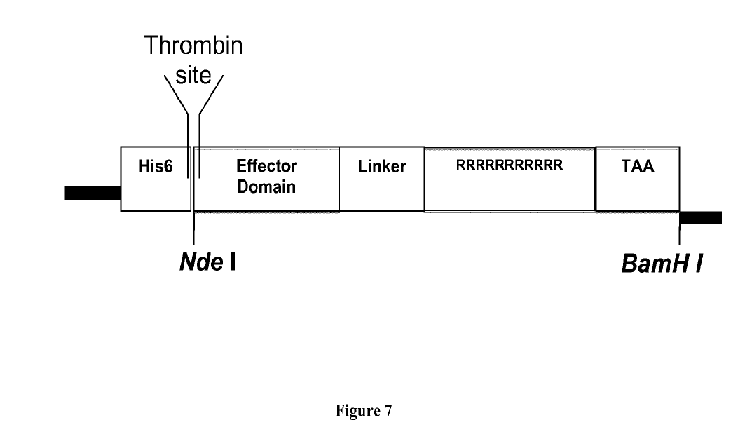

A poly-arginine protein transduction domain was fused respectively to the C-terminal of each reprogramming protein (Ngn3, PDX1 and MafA) through a linker (SEQ ID NO: 55) to form His6-Ngn3-11R, His6-PDX1-11R and His6-MafA-11R respectively (FIG. 7). His6 (SEQ ID NO: 59) was included to facilitate protein purification. These poly-arginine fused proteins were expressed in E. Coli in inclusion body form, which were then solubilized, refolded, and further purified to prepare transducible materials His6-Ngn3-11R, His6-PDX1-11R and His6-MafA-11R. The protein identities were confirmed by mass spectrometry and Western blot analysis.

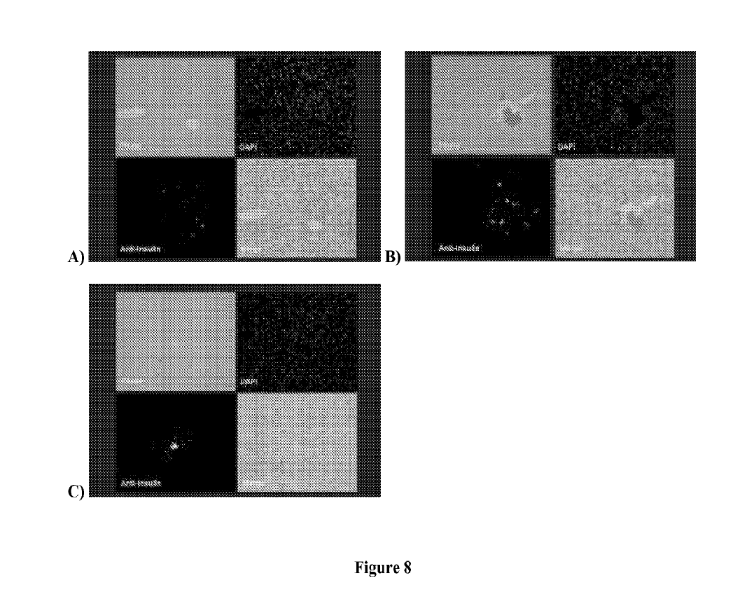

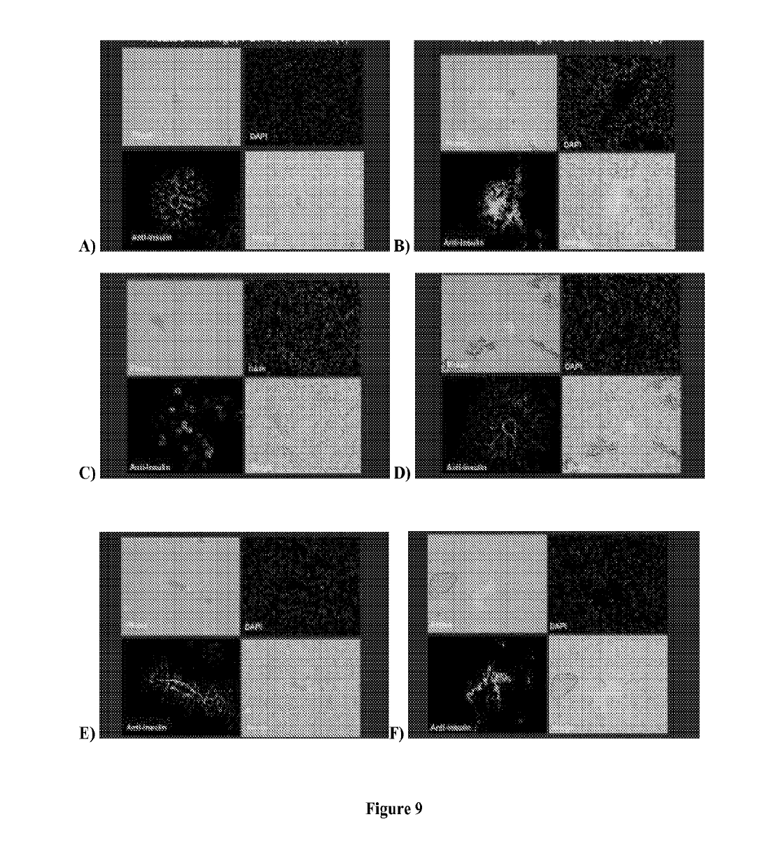

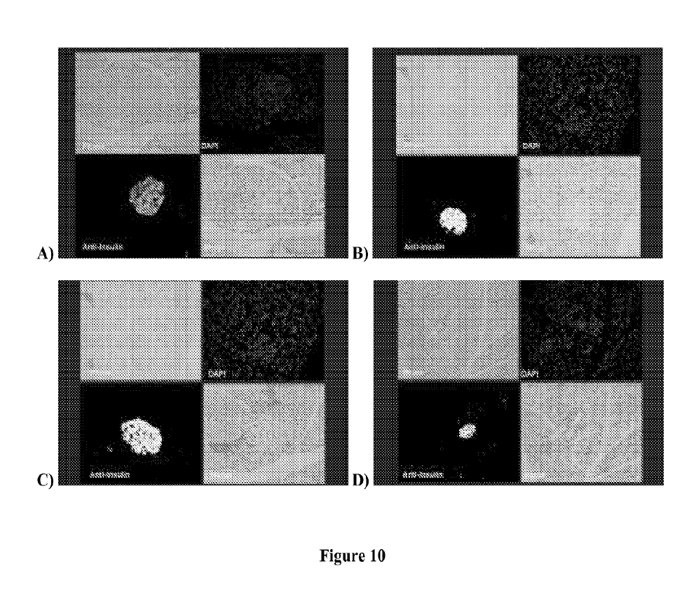

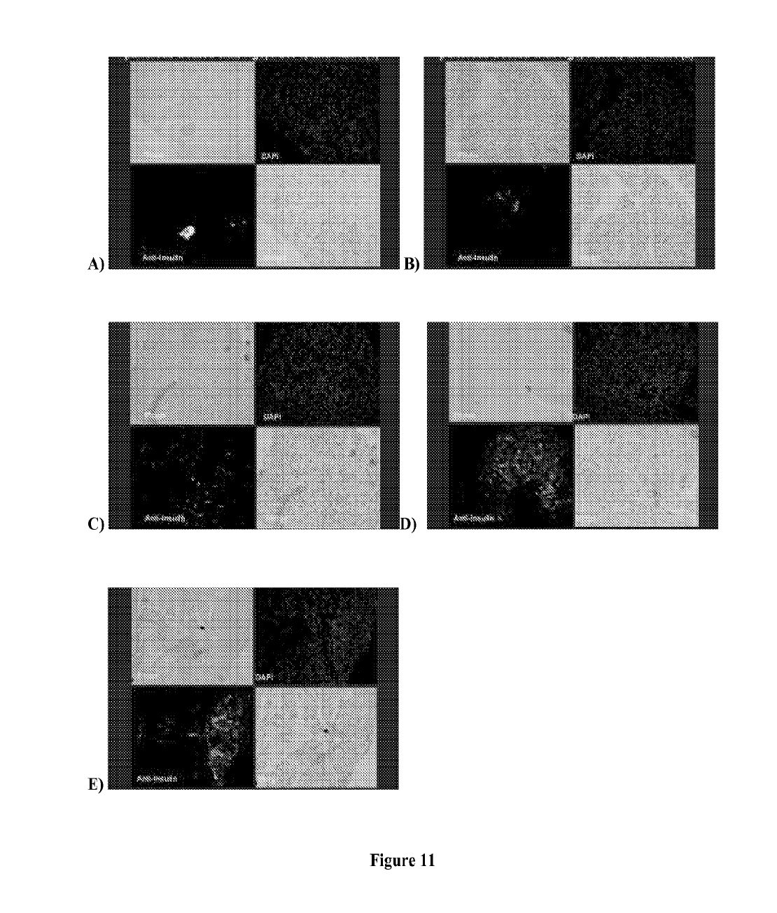

Six CD-1 mice (Charles River Laboratory) were divided into two groups: the treatment group and the control group. Transducible material His6-Ngn3-11R (1 mg/kg), His6-PDX1-11R (1 mg/kg), and His6-MafA-11R (1 mg/kg) were injected into each mouse by intraperitoneal (IP) in treatment group (Mouse-4, Mouse-5 and Mouse-6) and BSA (1 mg/kg) was injected into each mouse in the control group (Mouse-1, Mouse-2 and Mouse-3). There was no Greenish-brown or Yellow aspirate when needle penetrated into each mouse peritonea. Injections were repeated every day for 7 days. Mice of both treatment and control group were sacrificed on the 3rd day after the completion of all injections. The mouse liver and pancreas were washed with 1X PBS and fixed by 4% paraformaldehyde for overnight. Then the liver and pancreatic tissues were processed by standard Paraffin Embedding protocol. The Tissue sections, 5-micro in thickness, were prepared routinely with histology microtomes and mounted on standard histology glass slides. The wax in tissues was dissolved by xylene during processing of tissue sections. Tissue sectioning and histologic and immunohistochemical staining were performed using routine methods. For indirect fluorescent-antibody (IFA) assay, the slides were blocked with 0.05% Tween-20 (TBST) and 3% BSA for 1 hour at RT and were incubated with mouse anti-insulin antibody (Invitrogen) at 4° C. overnight. The slides were washed three times with PBS for 15 minutes at RT and incubated with fluorescein isothiocyanate (FITC) conjugated swine anti-mouse antibody (KPL) for 2 hours at RT. Same concentration of Mouse IgG was used as isotype control. Anti-DAPI antibody was added to slides as a nuclear marker. The slides were washed as before and mounted with aqueous mounting media (Biomeda, Foster City, CA). Endothelial markers were identified under the microscope (Olympus BX51, San Diego, CA) and merged cells were analyzed by Microsuite Biological Suite program (Olympus BX51, San Diego, CA) (FIGS. 8-11). The results showed that the treatment group had more insulin-producing cells (FIG. 9) in livers comparing to the control group (FIG. 8). The pancreas of the control group showed a cluster of insulin-producing cells (FIG. 10), while the pancreas of the treatment group showed insulin-producing cells in bigger area (FIG. 11). Therefore, the results showed that treatment of transducible materials His6-Ngn3-11R, His6-PDX1-11R, and His6-MafA-11R converted liver and/or pancreas cells to insulin-producing cells.

### Example 3

Reprogramming of T cells and programs them to Treg cells using transducible material Foxp3.

A poly-arginine protein transduction domain was fused to the C-terminal of each reprogramming protein Foxp3 through a linker (SEQ ID NO: 55) to form His6-Foxp3-11R (FIG. 7). His6 (SEQ ID NO: 59) was included to facilitate protein purification. The poly-arginine fused protein was expressed in E. Coli in inclusion body form, which were then solubilized, refolded, and further purified to prepare transducible materials His6-Foxp3-11R. The protein identities were confirmed by Western blot analysis.

100 ml of healthy human blood was collected from a donor and the peripheral blood mononuclear cells (PBMCs) were isolated by density-gradient centrifugation using Histopaque-1077 (Sigma-Aldrich, St Louis, Mo.). CD14+ monocytes were removed by magnetic bead selection (Miltenyi Biotec, Auburn, Calif.). Briefly, 108 PBMCs were incubated with 200 μL anti-CD14 microbeads (Miltenyi Biotec) in ice for 30 minutes. The cells were washed with cold 1X PBS with 2% FCS and centrifuged at 300 g for 10 minutes and then resuspended in 1X PBS with 2% FCS. The cell suspension was applied to the magnetic column and unbinding cells were passed through by washing 3 times with 1X PBS with 2% FCS. The PBMC/mono- were harvested by centrifuged at 300 g for 10 minutes.

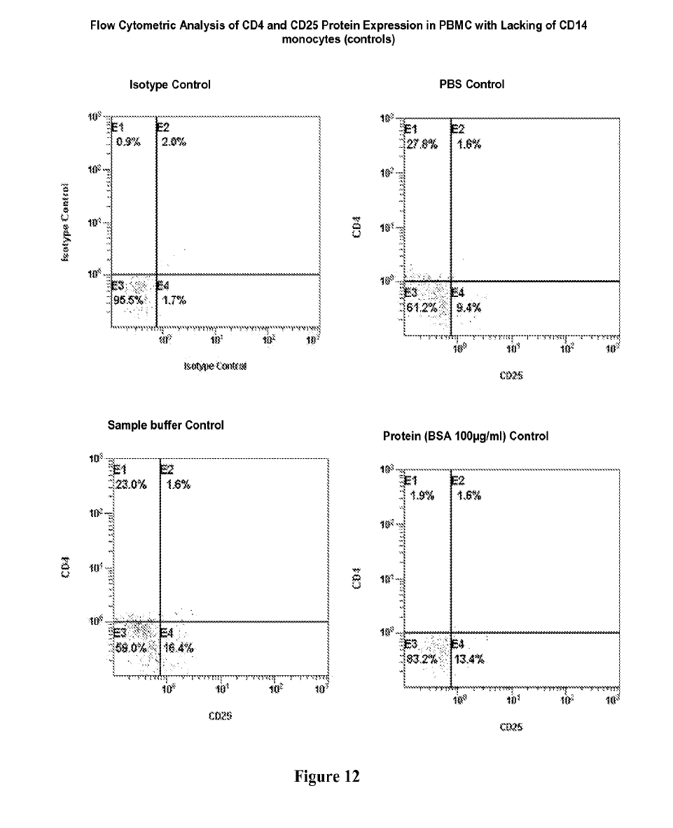

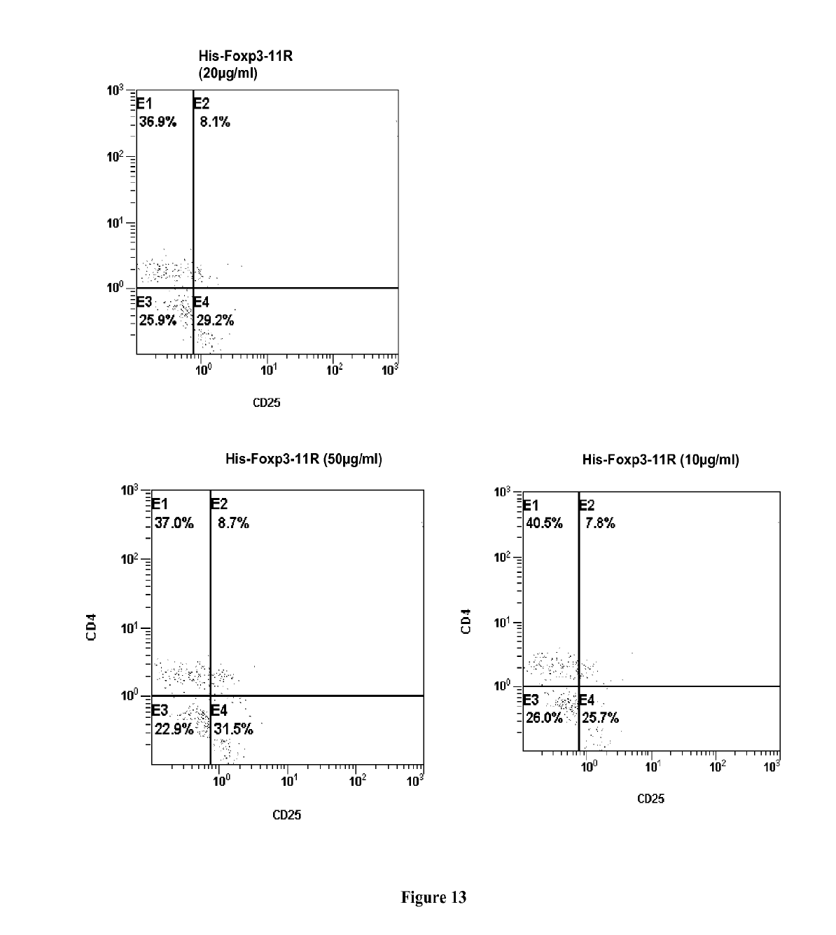

The PBMC/mono- were cultured in 6-well plates (Becton Dickinson, Gaithersburg, Md.) supplemented with 10% FBS, nonessential amino acids, 2 mM glutamine, 1 mM sodium pyruvate, 25 mM HEPES, 200 units/ml penicillin, and streptomycin at 37° C. and 5% CO₂. After 1 hour of culture, His6-Foxp3-11R (10 μg/ml, 20 μg/ml, or 50 μg/ml) was added to the cells. BSA (100 μg/ml) was added to another well as control. Same concentration of His6-Foxp3-11R or BSA was added after cultured for two days. After 5 days of culture, the cells were washed with PBS twice. The cells were re-suspended in 100 μL diluted and added rabbit anti-human CD25 for 90 minutes. The cells were washed three time with cold 1XPBS supplied 2% FBS and then the conjugated-PE mouse anti-human CD4 as well as conjugated-FITC goat anti-rabbit IgG were added to the cells for 60 minutes in ice. Conjugated-PE mouse IgG and rabbit IgG were incubated with another group cells as Isotype control. The cells were washed with PBS for flow cytometric analysis using a Beckman Coulter FC500 cytometer with Cytomics CXP software (Beckman Coulter, Fullerton, Calif.) (FIGS. 12 and 13). The results showed that the CD4+CD25+T cells (Treg cells) have dramatically increased with treatment of transducible material His6-Foxp3-11R, and the increase is protein-dose dependent.

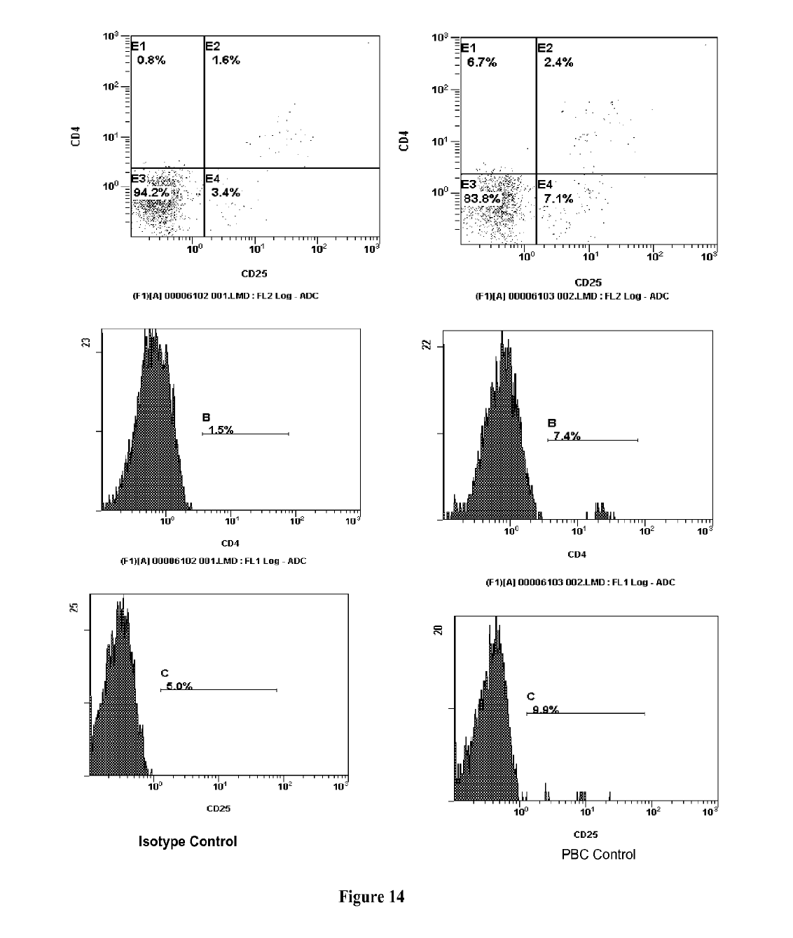

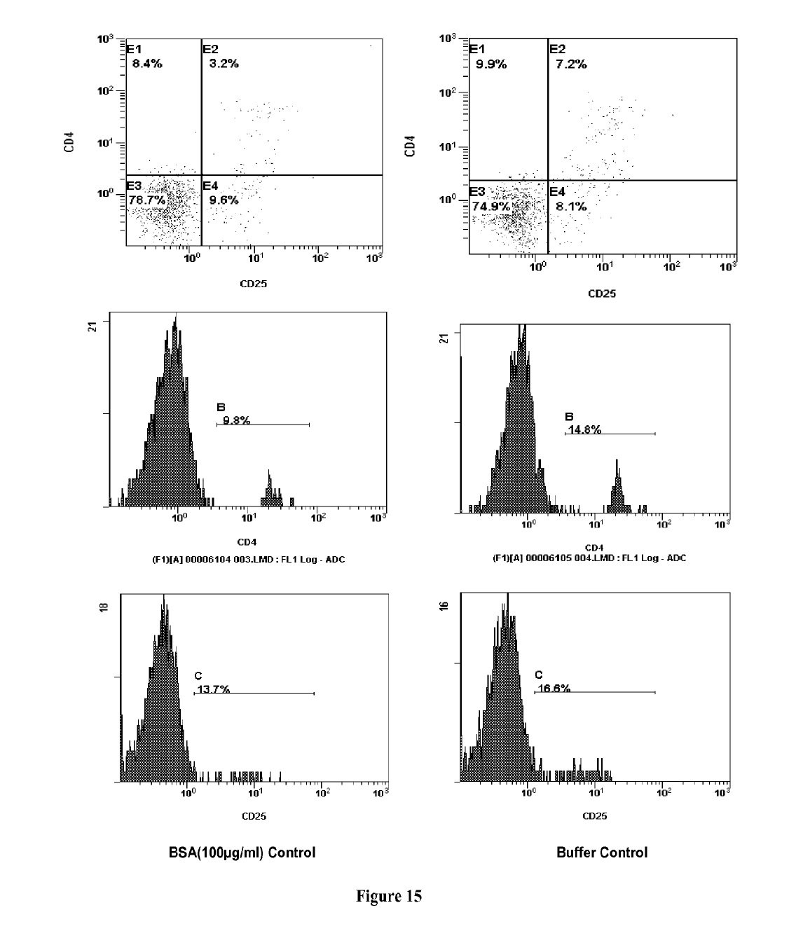

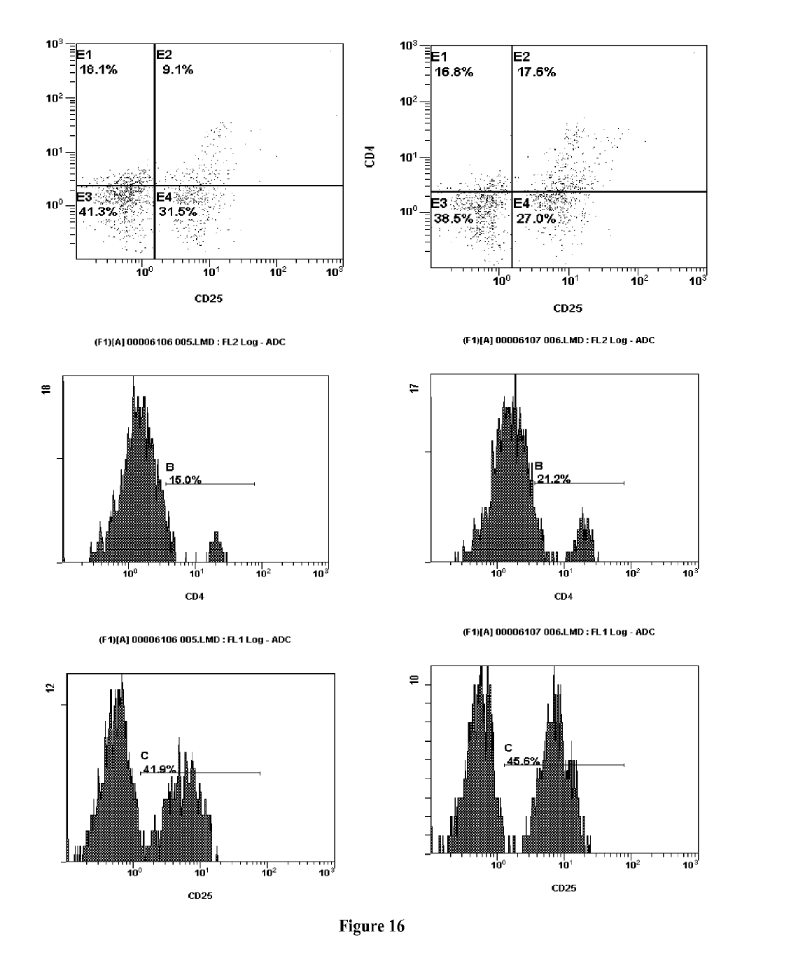

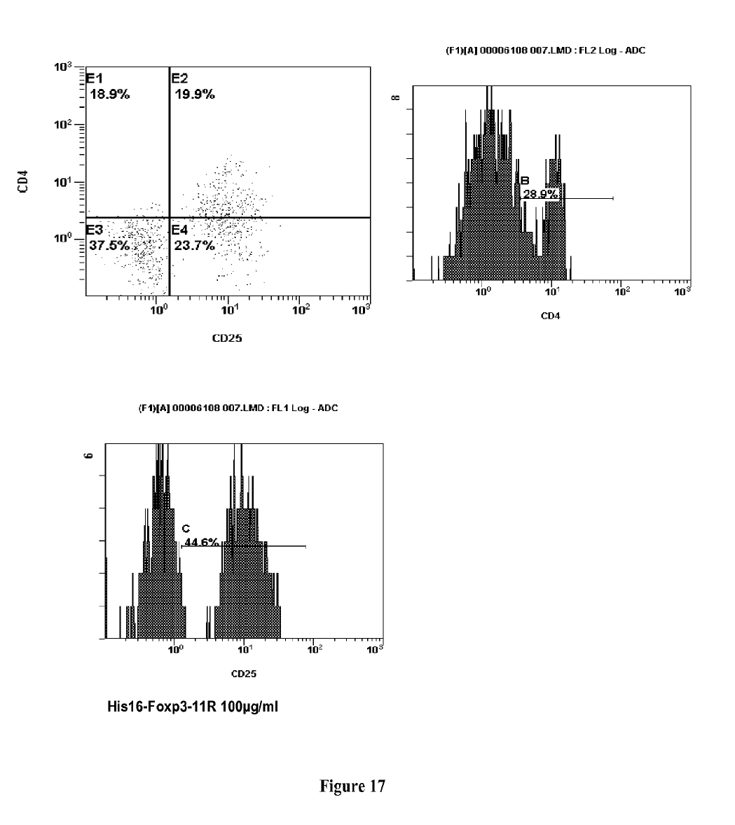

100 ml of healthy human blood was collected from a donor and the peripheral blood mononuclear cells (PBMCs) were isolated by density-gradient centrifugation using Histopaque-1077 (Sigma-Aldrich, St Louis, Mo.). The PBMC/mono were cultured in 6-well plates (Becton Dickinson, Gaithersburg, Md.) supplemented with 10% FBS, nonessential amino acids, 2 mM glutamine, 1 mM sodium pyruvate, 25 mM HEPES, 200 units/ml penicillin, and streptomycin at 37° C. and 5% CO2. After 1 hour of culture, Foxp3 (10 μg/ml, 50 μg/ml, 100 μg/ml) were added to the cells. BSA (100 μg/ml) was added to another well as control. Same concentration of the Foxp3 or BSA was added after cultured two days. Following 5 days of culture, the cells were washed with PBS twice. The cells were re-suspended in 100 μL diluted and added rabbit anti-human CD25 for 90 minutes. The cells were washed three time with cold 1XPBS supplied 2% FBS and then the conjugated-PE mouse anti-human CD4 as well as conjugated-FITC goat anti-rabbit IgG were added to the cells for 60minutes in ice. Conjugated-PE mouse IgG and rabbit IgG were incubated with another group cells as Isotype control. The cells were washed with PBS for flow cytometric analysis using a Beckman Coulter FC500 cytometer with Cytomics CXP software (Beckman Coulter, Fullerton, Calif.) (FIGS. 14-17). The results showed that the CD4+CD25+T cells (Treg cells) have dramatically increased with treatment of transducible material His6-Foxp3-11R, and the increase is protein-dose dependent.

1. A transducible material comprising an effector domain.

2. The transducible material of claim 1 further comprising a transduction domain.

3. The transducible material of claim 2, wherein the transduction domain is linked to the effector domain covalently, non-covalently or via a linker.

4. The transducible material of claim 1, wherein the effector domain is inherently transducible.

5. The transducible material of claim 1, wherein the effector domain is a polypeptide, a small molecule, or a polynucleotide.

6. The transducible material of claim 5 wherein the polypeptide is selected from the group consisting of Oct4, Sox2, K1f4, Lin28, Nanog, cMyc, Ngn3, PDX1, MafA, NeuroD, Foxp3, and any combination thereof.

7. The transducible material of claim 2, wherein the transduction domain is selected from the group consisting of a protein transduction domain, a cell penetrating peptide, a cell permeating peptide, an activatable cell penetrating peptide, a cell-targeting peptide and a polymer.

8. The transducible material of claim 7, wherein the protein transduction domain is selected from the group consisting of TAT, poly-arginine, Penetratin, Antennapedia, VP22, Transportan, MAP, MTS, PEP-1, Arg/Trp analogue, RRWRRWWRRWWRRW, polyguanidine peptoid, polyguanidine peptoid, inherent protein transduction domain, SEQ ID NO: 56, SEQ ID NO: 57, HIV-1 Rev, Flock house virus coat peptide, DNA-binding peptides, c-Fos, c-Jun and yeast GCN4.

9. The transducible material of claim 7, wherein the cell-targeting peptide is a peptides having an amino acid sequence selected from the group consisting of NGR, RGD, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 58.

10. The transducible material of claim 7, wherein the polymer is selected from the group consisting of a cationic lipid polymer and a nanoparticle.

11. The transducible material of claim 1, wherein the transducible material is capable of selectively transducing into one or more specific biological samples or capable of becoming transducible in a specific environment surrounding the biological sample.

12. The transducible material of claim 3, wherein the linker has an amino acid sequence set forth in SEQ ID: 55.

13. The transducible material of claim 1 further comprising one or more motifs that do not interrupt the function of the effector domain or the transduction domain.

14. The transducible material of claim 13 wherein the motif is covalently linked to the effector domain or the transduction domain.

15. The transducible material of claim 14, wherein the motif has an amino acid sequence set forth in SEQ ID: 59.

16. A method of reprogramming a biological sample, comprising:
exposing the biological sample to at least one transducible material of claim 1.

17. The method of claim 16 wherein the biological material is a cell, a tissue, or an organ from a biological organism.

18. The method of claim 17 wherein the biological organism is a microorganism, a plant or an animal.

19. The method of claim 16 wherein the biological sample is reprogrammed so as to cause proliferation, differentiation, transdifferentiation, retrodifferentiation, transdertermination, dedifferentiation, apoptosis or morphogenesis.

20. The method of claim 17 wherein the cell is reprogrammed to change from a first type cell to a second type cell.

21. The method of claim 20 wherein the first type cell is a somatic cell and the second type cell is a stem cell.

22. The method of claim 21, wherein the transducible materials are Oct4-11R, Sox2-11R, K1f4-11R and cMyc-11R.

23. The method of claim 20, wherein the first type cell is a hepatocyte or a pancreatic cell and the second type cell is an insulin producing cell.

24. The method of claim 23, wherein the transducible materials are His-Ngn3-11R, His6-PDX1-11R and His6-MafA-11R.

25. The method of claim 20, wherein the first type cell is a T cell and the second type cell is a regulatory T cell.

26. The method of claim 25, wherein the transducible material is His6-Foxp3-11R.

27. The method of claim 20 wherein the first type cell is a stem cell or a first progenitor cell and the second type cell is a second progenitor cell or a somatic cell.

28. A pharmaceutical composition comprising a transducible material of claim 1.

29. The pharmaceutical composition of claim 28 further comprising one or more growth factors.

30. The pharmaceutical composition of claim 29, wherein the growth factor is an islet growth factor.

31. A composition comprising a biological sample and a transducible material of claim 1, wherein the transducible material has transduced into the biological sample.

32. A method of treating a disease or condition in a biological organism comprising administering a pharmaceutical composition comprising a transducible material of claim 1 into the biological organism.

33. The method of claim 32 wherein the disease or condition is selected from the group consisting of tumor, cancer, metabolic disease, inflammatory condition, cardiac disease, neurogenerative disease, and autoimmune disease.

34. The method of claim 33, wherein the metabolic diseases or conditions are diabetes.

35. The method of claim 33, wherein the neurogenerative disease is selected from the group consisting of Alzheimer's disease, amyotrophic lateral sclerosis (Lou Gehrig's Disease, Huntington's disease, and Parkingson's disease.

36. The method of claim 33, wherein the inflammatory disease is selected from the group consisting of anemia, spinal cord injury, burns, and arthritis.

37. The method of claim 33, wherein the autoimmune disease is selected from the group consisting of acute disseminated encephalomyelitis, Addison's disease, alopecia areata, ankylosing spondylitis, antiphospholipid antibody syndrome, anemia, autoimmune hemolytic anemia, pernicious anaemia, arthritis, psoriatic arthritis, rheumatoid arthritis, diabetes mellitus type 1, autoimmune hepatitis, autoimmune inner ear disease, bullous pemphigoid, coeliac disease, Chagas disease, chronic obstructive pulmonary disease, Crohns disease, dermatomyositis, endometriosis, Goodpasture's syndrome, Graves' disease, Guillain-Barre syndrome GBS, Hashimoto's disease, hidradenitis suppurativa, Kawasaki disease, IgA nephropathy, idiopathic thrombocytopenic purpura, interstitial cyctitis, lupus erythematosus, mixed connective tissue disease, morphea, multiple sclerosis, myasthenia gravis, narcolepsy, neuromyotonia, pemphigus vulgaris, psoriasis, polymyositis, primary billiary cirrhosis, schizophrenia, scleroderma, Sjogren's syndrome, stiff person syndrome, temporal arteritis, ulcerative colitis, vasculitis, vitiligo, and Wegener's granulomatosis.

38. A method of treating a disease or condition of a biological organism comprising:
removing a biological sample from the biological organism;
exposing the biological sample to a transducible material of claim 1; and
transplanting the biological sample transduced with the transducible material back to the biological organism.

39. A method of developing cell-based therapies for various diseases or conditions comprising:
reprogramming an iPSC, an embryonic stem cell, or a progenitor cell to a transplantable somatic cell or a transplantable progenitor cell by exposing the iPSC, the embryonic stem cell or the progenitor cell to at least one transducible material of claim 1;
transplanting the transplantable somatic or progenitor cell into a biological sample or a biological organism; and
assessing the therapeutic effects of the transplantable somatic or progenitor cell.

40. A method of developing a disease model comprising:
exposing an iPSC, an embryonic stem cell, or a progenitor cell to at least one transducible material of claim 1 so as to reprogram into a transplantable somatic cell or a transplantable progenitor cell;
transplanting the transplantable somatic or progenitor cell into a biological organism; and
developing a disease model having the transplantable somatic or progenitor cell.

41. A method of a developing disease model comprising:
exposing a patient-specific cell to at least one transducible material of claim 1 so as to reprogram into a patient specific iPSC;
generating a derived cell from the iPSC with or without the transducible materials; and
developing a disease model using the patient-specific iPSC or iPSC-derived cell.

42. A method of identify an effector domain comprising:
covalently linking a test effector domain to a transduction domain to form a test transducible molecule;
exposing the test molecule to a biological sample, and
measuring a reprogramming level of the biological sample.

43. A method of developing drug screening or toxicity models comprising:
reprogramming a somatic cell, a progenitor cell, or a multipotent cell to an iPSC via exposing to at least one transducible material of claim 1;
generating a derived cell from the iPSC with or without exposing to the transducible materials; and
using the iPSC and/or the iPSC-derived cell to screen the effects and/or toxicities of different compounds.


SEQUENCE LISTING (64 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 3 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 4 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 5 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 6 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 7 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 8 - protein, 214 aa, Homo sapiens
MTPQPSGAPTVQVTRETERSFPRASEDEVTCPTSAPPSPTRTPGNCAEAEEGGCRGAPRKLRARRGGRSRPKSELALSKQRRSRRKKANDRERNRMHDLNSALDALRGVLPTFPDDAKLTKIETLRFAHNYIWALTQTLRIADHSLYALEPPAPHCGELGSPGGPPGDWGSLYSPVSQAGSLSPAASLEERPGLLGATSSACLSPGSLAFSDFL

SEQ ID NO: 9 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 10 - protein, 352 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 11 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 12 - protein, 380 aa, Artificial Sequence
  transducible material
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNESGGGGSPGRRRRRRRRRRR

SEQ ID NO: 13 - protein, 337 aa, Artificial Sequence
  transducible material
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMESGGGGSPGRRRRRRRRRRR

SEQ ID NO: 14 - protein, 490 aa, Artificial Sequence
  transducible material
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFESGGGGSPGRRRRRRRRRRR

SEQ ID NO: 15 - protein, 474 aa, Artificial Sequence
  transducible material
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAESGGGGSPGRRRRRRRRRRR

SEQ ID NO: 16 - protein, 229 aa, Artificial Sequence
  transducible material
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNESGGGGSPGRRRRRRRRRRR

SEQ ID NO: 17 - protein, 325 aa, Artificial Sequence
  transducible material
MSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVESGGGGSPGRRRRRRRRRRR

SEQ ID NO: 18 - protein, 376 aa, Artificial Sequence
  transducible material
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHDESGGGGSPGRRRRRRRRRRR

SEQ ID NO: 19 - protein, 234 aa, Artificial Sequence
  transducible material
MTPQPSGAPTVQVTRETERSFPRASEDEVTCPTSAPPSPTRTPGNCAEAEEGGCRGAPRKLRARRGGRSRPKSELALSKQRRSRRKKANDRERNRMHDLNSALDALRGVLPTFPDDAKLTKIETLRFAHNYIWALTQTLRIADHSLYALEPPAPHCGELGSPGGPPGDWGSLYSPVSQAGSLSPAASLEERPGLLGATSSACLSPGSLAFSDFLESGGGGSPGRRRRRRRRRRR

SEQ ID NO: 20 - protein, 303 aa, Artificial Sequence
  transducible material
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPRESGGGGSPGRRRRRRRRRRR

SEQ ID NO: 21 - protein, 372 aa, Artificial Sequence
  transducible material
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFLESGGGGSPGRRRRRRRRRRR

SEQ ID NO: 22 - protein, 451 aa, Artificial Sequence
  transducible material
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPESGGGGSPGRRRRRRRRRRR

SEQ ID NO: 23 - protein, 400 aa, Artificial Sequence
  transducible material
MGSSHHHHHHSSGLVPRGSHMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNESGGGGSPGRRRRRRRRRRR

SEQ ID NO: 24 - protein, 357 aa, Artificial Sequence
  transducible material
MGSSHHHHHHSSGLVPRGSHMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMESGGGGSPGRRRRRRRRRRR

SEQ ID NO: 25 - protein, 510 aa, Artificial Sequence
  transducible material
MGSSHHHHHHSSGLVPRGSHMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFESGGGGSPGRRRRRRRRRRR

SEQ ID NO: 26 - protein, 494 aa, Artificial Sequence
  transducible material
MGSSHHHHHHSSGLVPRGSHMDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAESGGGGSPGRRRRRRRRRRR

SEQ ID NO: 27 - protein, 249 aa, Artificial Sequence
  transducible material
MGSSHHHHHHSSGLVPRGSHMGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNESGGGGSPGRRRRRRRRRRR

SEQ ID NO: 28 - protein, 345 aa, Artificial Sequence
  transducible material
MGSSHHHHHHSSGLVPRGSHMSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVESGGGGSPGRRRRRRRRRRR

SEQ ID NO: 29 - protein, 396 aa, Artificial Sequence
  transducible material
MGSSHHHHHHSSGLVPRGSHMTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHDESGGGGSPGRRRRRRRRRRR

SEQ ID NO: 30 - protein, 254 aa, Artificial Sequence [sequence omitted]

SEQ ID NO: 31 - protein, 323 aa, Artificial Sequence
  transducible material
MGSSHHHHHHSSGLVPRGSHMNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPRESGGGGSPGRRRRRRRRRRR

SEQ ID NO: 32 - protein, 392 aa, Artificial Sequence
  transducible material
MGSSHHHHHHSSGLVPRGSHMAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFLESGGGGSPGRRRRRRRRRRR

SEQ ID NO: 33 - protein, 471 aa, Artificial Sequence
  transducible material
MGSSHHHHHHSSGLVPRGSHMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPESGGGGSPGRRRRRRRRRRR

SEQ ID NO: 34 - protein, 11 aa, Human immunodeficiency virus type 1
YGRKKRRQRRR

SEQ ID NO: 35 - protein, 10 aa, Artificial Sequence
  protein transducible domain
RRRRRRRRRR

SEQ ID NO: 36 - protein, 11 aa, Artificial Sequence
  protein transducible domain
RRRRRRRRRRR

SEQ ID NO: 37 - protein, 20 aa, Artificial Sequence
  protein transducible domain
ESGGGGSPGRRRRRRRRRRR

SEQ ID NO: 38 - protein, 16 aa, Drosophila melanogaster
RQIKIWFQNRRMKWKK

SEQ ID NO: 39 - protein, 34 aa, herpes simplex virus 7
DAATATRGRSAASRPTQRPRAPARSASRPRRPVQ

SEQ ID NO: 40 - protein, 27 aa, Artificial Sequence
  protein transducible domain
GWTLNSAGYLLGKINLKALAALAKKIL

SEQ ID NO: 41 - protein, 18 aa, Artificial Sequence
  protein transducible domain
KLALKLALKALKAALKLA

SEQ ID NO: 42 - protein, 15 aa, Artificial Sequence
  protein transducible domain
AAVALLPAVLLALLP

SEQ ID NO: 43 - protein, 21 aa, Artificial Sequence
  protein transducible domain
KETWWETWWTEWSQPKKKRKV

SEQ ID NO: 44 - protein, 14 aa, Artificial Sequence
  protein transducible domain
RRWRRWWRRWWRRW

SEQ ID NO: 45 - protein, 12 aa, Artificial Sequence
  cell-targeting peptide
TSPLNIHNGQKL

SEQ ID NO: 46 - protein, 5 aa, Artificial Sequence
  cell-targeting peptide
CGKRK

SEQ ID NO: 47 - protein, 9 aa, Artificial Sequence
  cell-targeting peptide
CGNKRTRGC

SEQ ID NO: 48 - protein, 7 aa, Artificial Sequence
  cell-targeting peptide
SMSIARL

SEQ ID NO: 49 - protein, 7 aa, Artificial Sequence
  cell-targeting peptide
FQHPSFI

SEQ ID NO: 50 - protein, 7 aa, Artificial Sequence
  cell-targeting peptide
VHSPNKK

SEQ ID NO: 51 - protein, 6 aa, Artificial Sequence
  cell-targeting peptide
RRPYIL

SEQ ID NO: 52 - protein, 12 aa, Artificial Sequence
  cell-targeting peptide
EDYELMDLLAYL

SEQ ID NO: 53 - protein, 7 aa, Artificial Sequence
  cell-targeting peptide
LTVSPWY

SEQ ID NO: 54 - protein, 7 aa, Artificial Sequence
  cell-targeting peptide
ATWLPPR

SEQ ID NO: 55 - protein, 9 aa, Artificial Sequence
  linker
ESGGGGSPG

SEQ ID NO: 56 - protein, 16 aa, Homo sapiens
RHIKIWFQNRRMKWKK

SEQ ID NO: 57 - protein, 34 aa, Homo sapiens
KPKRRGPKKKKMTKARLERFKLRRMKANARERNR

SEQ ID NO: 58 - protein, 10 aa, Homo sapiens
EHWSYGLRPG

SEQ ID NO: 59 - protein, 20 aa, Artificial Sequence
  protein purification
MGSSHHHHHHSSGLVPRGSH

SEQ ID NO: 60 - protein, 17 aa, Human immunodeficiency virus type 1
TRQARRNRRRRWRERQR

SEQ ID NO: 61 - protein, 15 aa, flock house virus
RRRRNRTRRNRRRVR

SEQ ID NO: 62 - protein, 26 aa, Homo sapiens
KRRIRRERNKMAAAKSRNRRRELTDT

SEQ ID NO: 63 - protein, 28 aa, Homo sapiens
RIKAERKRMRNRIAASKSRKRKLERIAR

SEQ ID NO: 64 - protein, 22 aa, Artificial Sequence
  yease GCN4 peptide
KRARNTEAARRSRARKLQRMKQ